(19) United States
(12) Patent Application Publication
(10) Pub. No.: US 2023/0190132 A1
(43) Pub. Date: Jun. 22, 2023

(54) PATIENT SUPPORT APPARATUS WITH SPECTROMETER

(71) Applicant: Stryker Corporation, Kalamazoo, MI (US)

(72) Inventor: Jerald A. Trepanier, Kalamazoo, MI (US)

(21) Appl. No.: 17/854,036

(22) Filed: Jun. 30, 2022

Related U.S. Application Data
(60) Provisional application No. 63/290,194, filed on Dec. 16, 2021.

(51) Int. Cl. A61B 5/083; A61B 5/00; A61B 5/11; A61B 5/087; A61G 7/00

(52) U.S. Cl. CPC A61B 5/0836; A61B 5/746; A61B 5/1113; A61B 5/0873; A61B 5/1128; A61G 7/00; A61B 2560/0223; A61B 2562/0247; A61B 2560/0242

(57) ABSTRACT

A patient support apparatus, such as a bed, cot, stretcher, or the like, includes a frame, a support surface, an emitter, a detector, and a controller. The emitter and detector are coupled to the patient support apparatus at first and second locations, respectively. The emitter is adapted to emit electromagnetic waves in a direction aimed toward the detector. The detector is adapted to detect the electromagnetic waves emitted from the emitter. The controller is adapted to perform a spectral analysis of the detected electromagnetic waves to determine a parameter associated with the gas exhaled by a patient positioned on the patient support apparatus. The parameter may refer to a carbon dioxide level of gas exhaled by the patient, the patient's respiration rate, the patient's metabolic rate, or the like. The emitter, detector, and controller may alternatively be separate from, but attachable to, and in communication with, the patient support apparatus.

### CROSS-REFERENCE TO RELATED APPLICATIONS

This application claims priority to U.S. provisional patent application Ser. No. 63/290,194 filed Dec. 16, 2021, by inventor Jerald Trepanier and entitled PATIENT SUPPORT APPARATUS WITH SPECTROMETER, the complete disclosure of which is incorporated herein by reference.

### BACKGROUND

The present disclosure relates to patient support apparatuses, such as beds, stretcher, cots, and the like.

Patients in healthcare facilities such as hospitals, or the like, are generally assigned to a bed or stretcher while they are present in the healthcare facility. Often, it is desirable to be able to remotely monitor one or more physiological aspects of the patient health while he or she is resting on his or her assigned patient support apparatus, thereby enabling caregivers to be apprised of those physiological aspects while they are positioned elsewhere in the healthcare facility.

### SUMMARY

According to various aspects of the present disclosure, a patient support apparatus is provided that includes a built-in spectrometer that is adapted to perform a spectral analysis of the gas exhaled by the patient while he or she is positioned on the patient support apparatus. The spectral analysis may be used to determine one or more parameters of the patient and/or the exhaled gas, such as the respiration rate of the patient, the metabolic rate of the patient, the quantity and/or concentration of carbon dioxide in the exhaled gas, whether the patient is awake or asleep, and/or other parameters. In some aspects, data derived from the spectral analysis is communicated by the patient support apparatus to a remote server and automatically associated with the patient who is assigned to that particular patient support apparatus, thereby relieving the caregiver of the need to manually associate the data of the spectrometer with a particular patient. In other aspects, the spectrometer is physically separable from the patient support apparatus, but adapted to be coupled thereto, and to communicate with the patient support apparatus. Still other features and aspects of the present disclosure will be apparent to one of ordinary skill in the art from the following written description and accompanying drawings.

A patient support apparatus according to a first aspect of the present disclosure includes a frame, a support surface, an emitter, a detector, and a controller. The support surface is supported on the frame and adapted to support a patient thereon. The emitter is coupled to a first location on the patient support apparatus and adapted to emit electromagnetic waves. The detector is coupled to a second location on the patient support apparatus and adapted to detect the electromagnetic waves emitted by the emitter. The controller is adapted to perform a spectral analysis of the electromagnetic waves detected by the detector to determine a parameter associated with the gas exhaled by the patient when the patient is positioned on the patient support apparatus.

According to other aspects of the present disclosure, the parameter includes at least one of the following: a level of carbon dioxide exhaled by the patient, a breathing rate of the patient, or a metabolic rate of the patient.

In those aspects where the controller is adapted to determine a metabolic rate of the patient, the patient support apparatus may further include a plurality of force sensors adapted to detect a weight of the patient when the patient is positioned on the support surface. The controller may further be adapted to use the weight of the patient when determining the metabolic rate.

In some aspects, the patient support apparatus further includes a transceiver adapted to communicate with a remote server, and the controller is adapted to transmit an alert to the remote server if the metabolic rate is at least one of the following: less than a lower threshold or greater than an upper threshold.

In some aspects, the patient support apparatus further comprises a transceiver adapted to communicate with a remote server, and the controller is adapted to transmit the parameter to the remote server.

A camera is included, in some aspects, and communicates with the controller. In such aspects, the controller is further adapted to process an image captured by the camera to determine a relative position of the patient's face to the detector.

The controller, in some aspects, is further adapted to take calibration readings from the detector when no patient is present on the patient support apparatus.

The patient support apparatus further includes, in some aspects, a plurality of force sensors adapted to detect a weight of the patient when the patient is positioned on the support surface, and the controller is further adapted to not take the calibration readings if patient weight is detected by the plurality of force sensors.

In some aspects, the patient support apparatus further includes a plurality of detectors, and the controller is further adapted to select at least one of the plurality of detectors based on its relative position to the patient's face. The controller is further adapted to perform the spectral analysis on the electromagnetic waves detected by the selected detector(s).

In some aspects, the patient support apparatus further includes a location receiver adapted to receive a location ID from a fixed locator unit positioned within a healthcare facility in which the patient support apparatus is located. The location receiver is adapted to receive the location ID when the patient support apparatus is positioned adjacent the fixed locator unit. The location receiver may be an infrared receiver and the location ID may be sent from the fixed locator unit to the location receiver via infrared waves.

In some aspects, the patient support apparatus further includes a transceiver adapted to communicate with a remote server, and the controller is adapted to transmit the location ID and the parameter to the remote server.

In some aspects, the patient support apparatus further includes a plurality of detectors coupled to the patient support apparatus, and the emitter is adapted to change an aim of the electromagnetic waves such that the electromagnetic waves are aimed at different ones of the plurality of detectors at different times.

The patient support apparatus, in some aspects, includes first and second siderails positioned on first and second sides of the support surface, respectively, and the first location is on the first siderail and the second location is on the second siderail.

In some aspects, the patient support apparatus further includes a plurality of detectors coupled to the second siderail, and the emitter is adapted to change an aim of the electromagnetic waves such that the electromagnetic waves are aimed at different ones of the plurality of detectors at different times.

In some aspects, the electromagnetic waves are infrared waves.

The controller, in some aspects, is adapted to determine a baseline level of carbon dioxide in a sample of air near the patient support apparatus from the calibration readings.

The controller, in some aspects, is further adapted to use the baseline level of carbon dioxide in the air sample to determine a level of carbon dioxide exhaled by the patient when the patient is positioned on the patient support apparatus.

According to another aspect of the present disclosure, a spectrometer is provided that includes an emitter, a detector, a transmitter, and a controller. The spectrometer is adapted to be attached to, and communicate with, a patient support apparatus. The emitter is adapted to be coupled to a first location on the patient support apparatus and to emit electromagnetic waves. The detector is adapted to be coupled to a second location on the patient support apparatus and to detect the electromagnetic waves emitted by the emitter. The controller is adapted to perform a spectral analysis of the electromagnetic waves detected by the detector to determine a parameter associated with gas exhaled by the patient when the patient is positioned on the patient support apparatus. The controller is further adapted to use the transmitter to send data to a receiver integrated into the patient support apparatus.

In some aspects, the parameter is at least one of a level of carbon dioxide exhaled by the patient, a breathing rate of the patient, or a metabolic rate of the patient.

The spectrometer, in some aspects, further includes a transceiver adapted to communicate with a remote server, and the controller is adapted to transmit the parameter to the remote server.

In some aspects, the spectrometer includes a camera in communication with the controller, and the controller is further adapted to process an image captured by the camera to determine a relative position of the patient's face to the detector.

In some aspects, the spectrometer includes a receiver adapted to receive a signal from the patient support apparatus indicating that no patient weight is detected on the patient support apparatus, wherein the controller is further adapted to take calibration readings from the detector when no patient is detected on the patient support apparatus.

In some aspects, the controller is further adapted to not take the calibration readings if patient weight is detected by the patient support apparatus.

The patient support apparatus, in some aspects, further includes a receiver adapted to receive a signal from the patient support apparatus indicating a weight of the patient on the patient support apparatus, and the controller is further adapted to use the weight of the patient when determining the metabolic rate.

The spectrometer, in some aspects, includes a transceiver adapted to communicate with a remote server, and the controller is adapted to transmit an alert to the remote server if the metabolic rate is at least one the following: less than a lower threshold or greater than an upper threshold.

In some aspects, the spectrometer further includes a plurality of detectors, and the controller is adapted to select at least one of the plurality of detectors based on the relative position of the patient's face to the detector. The controller is further adapted to perform the spectral analysis on the electromagnetic waves detected by the selected at least one of the detectors.

The spectrometer, in some aspects, further includes a plurality of detectors adapted to be coupled to the patient support apparatus, and the emitter is adapted to change an aim of the electromagnetic waves such that the electromagnetic waves are aimed at different ones of the plurality of detectors at different times.

In some aspects, the first location is a first siderail positioned adjacent a first side of the patient support apparatus, and the second location is a second siderail positioned adjacent a second side of the patient support apparatus.

The spectrometer, in some aspects, includes a plurality of detectors adapted to be coupled to the second siderail, and the emitter is adapted to change an aim of the electromagnetic waves such that the electromagnetic waves are aimed at different ones of the plurality of detectors at different times.

The electromagnetic waves, in some aspects, are infrared waves.

The controller, in some aspects, is adapted to determine a baseline level of carbon dioxide in a sample of air near the patient support apparatus from the calibration readings.

The controller, in some aspects, may further be adapted to use the baseline level of carbon dioxide in the air sample to determine a level of carbon dioxide exhaled by the patient when the patient is positioned on the patient support apparatus.

In some aspects, the data sent to the patient support apparatus includes at least one of the following: the parameter, a unique identifier of the spectrometer, or data regarding the parameter.

The transmitter, in some aspects, is adapted to communicate directly with the receiver of the patient support apparatus without communicating with any intermediate structures.

The patient support apparatus, in some embodiments, includes a nurse call control adapted to transmit a signal to an off-board nurse call system when the patient activates the nurse call control. A microphone onboard the patient support apparatus allows the user to speak with a remotely positioned nurse.

In some aspects, the patient support apparatus is a hospital bed.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first embodiment of the present disclosure;

FIG. 2 is a perspective view of a litter frame and a pair of lift header assemblies of the patient support apparatus;

FIG. 3 is a perspective view of a base and a pair of lifts of the patient support apparatus;

FIG. 4 is a diagram of the patient support apparatus, a wireless headwall unit, and a wireless communication link between the patient support apparatus and a wall outlet of a room within a healthcare facility;

FIG. 5 is a block diagram of several of the components of the patient support apparatus and the wireless headwall unit of FIG. 4;

FIG. 6 is a plan view of the head section of the patient support apparatus illustrating a beams of electromagnetic waves emitted by a spectrometer;

FIG. 7 is a side view of the head section of the patient support apparatus illustrating a plurality of detectors of the spectrometer of FIG. 6; and

FIG. 8 is a perspective view of a stand-alone spectrometer that may be detachably coupled to a patient support apparatus.

### DETAILED DESCRIPTION OF THE EMBODIMENTS

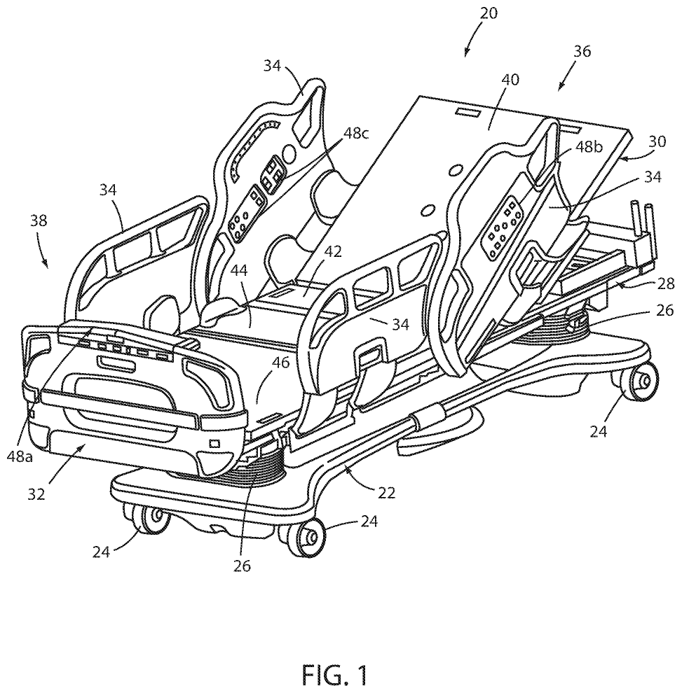

An illustrative patient support apparatus 20 according to a first embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, a chair, or the like.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a footboard 32 and a plurality of siderails 34. Siderails 34 are all shown in a raised position in FIG. 1 but are each individually movable to a lower position in which ingress into, and egress out of, patient support apparatus 20 is not obstructed by the lowered siderails 34.

Lifts 26 are adapted to raise and lower litter frame 28 with respect to base 22. Lifts 26 may be hydraulic actuators, pneumatic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted. That is, litter frame 28 includes a head end 36 and a foot end 38, each of whose height can be independently adjusted by the nearest lift 26. Patient support apparatus 20 is designed so that when an occupant lies thereon, his or her head will be positioned adjacent head end 36 and his or her feet will be positioned adjacent foot end 38.

Litter frame 28 provides a structure for supporting support deck 30, footboard 32, and siderails 34. Support deck 30 provides a support surface for a mattress (not shown in FIG. 1), such as, but not limited to, an air, fluid, or gel mattress. Alternatively, another type of soft cushion may be supported on support deck 30 so that a person may comfortably lie and/or sit thereon. The top surface of the mattress or other cushion forms a support surface for the occupant.

Support deck 30 is made of a plurality of sections, some of which are pivotable about generally horizontal pivot axes. In the embodiment shown in FIG. 1, support deck 30 includes a head section 40, a seat section 42, a thigh section 44, and a foot section 46. Head section 40, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 44 and foot section 46 may also be pivotable about generally horizontal pivot axes.

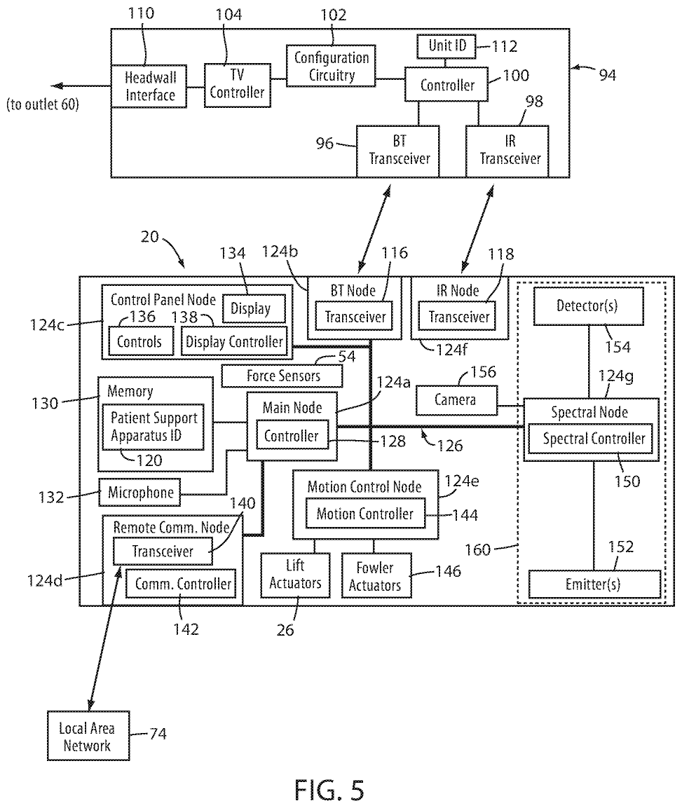

Patient support apparatus 20 further includes a plurality of control panels 48 that enable a user of patient support apparatus 20, such as a patient and/or an associated caregiver, to control one or more aspects of patient support apparatus 20. In the embodiment shown in FIG. 1, patient support apparatus 20 includes a footboard control panel 48a, a pair of outer side rail control panels 48b (only one of which is visible), and a pair of inner side rail control panels 48c (only one of which is visible). Footboard control panel 48a and outer side rail control panels 48b are intended to be used by caregivers, or other authorized personnel, while inner side rail control panels 48c are intended to be used by the patient associated with patient support apparatus 20. Each of the control panels 48 includes a plurality of controls 136 (FIG. 5), although each control panel 48 does not necessarily include the same controls and/or functionality.

Among other functions, controls 136 (FIG. 5) of control panel 48a allow a user to control one or more of the following: change a height of litter frame 28, raise or lower head section 40, activate and deactivate a brake for wheels 24, arm and disarm an exit detection system, control a spectrometer coupled to patient support apparatus 20 (as will be discussed in more detail below), and perform other tasks. One or both of the inner siderail control panels 48c also include at least one control that enables a patient to call a remotely located nurse (or other caregiver). In addition to the nurse call control, one or both of the inner siderail control panels 48c also include one or more controls for controlling one or more features of one or more room devices positioned within the same room as the patient support apparatus 20. As will be described in more detail below, such room devices include, but are not necessarily limited to, a television, a reading light, and/or a room light. With respect to the television, the features that may be controllable by one or more controls on control panel 48c include, but are not limited to, the volume, the channel, the closed-captioning, and/or the power state of the television. With respect to the room and/or night lights, the features that may be controlled by one or more controls on control panel 48c include the on/off state and/or the brightness level of these lights.

Footboard control panel 48a is implemented in the embodiment shown in FIG. 1 as a control panel having a lid (flipped down in FIG. 1) underneath which is positioned a plurality of controls. As with all of the controls of the various control panels 48, the controls of control panel 48a may be implemented as buttons, dials, switches, or other devices. Any of control panels 48a-c may also include a display for displaying information regarding patient support apparatus 20. The display is a touchscreen in some embodiments.

In some embodiments, footboard control panel 48a may take on the form of the footboard control panel 48a disclosed in commonly assigned PCT patent application serial number PCT/US2021/32426 filed May 14, 2021, by applicant Stryker Corporation and entitled PATIENT SUPPORT APPARATUSES WITH HEADWALL COMMUNICATION, the complete disclosure of which is incorporated herein by reference. Additionally, or alternatively, patient control panel 48c may take on the form of the patient control panel 48c disclosed in the aforementioned PCT patent application. Other types of footboard control panels 48a-c may, of course, be implemented.

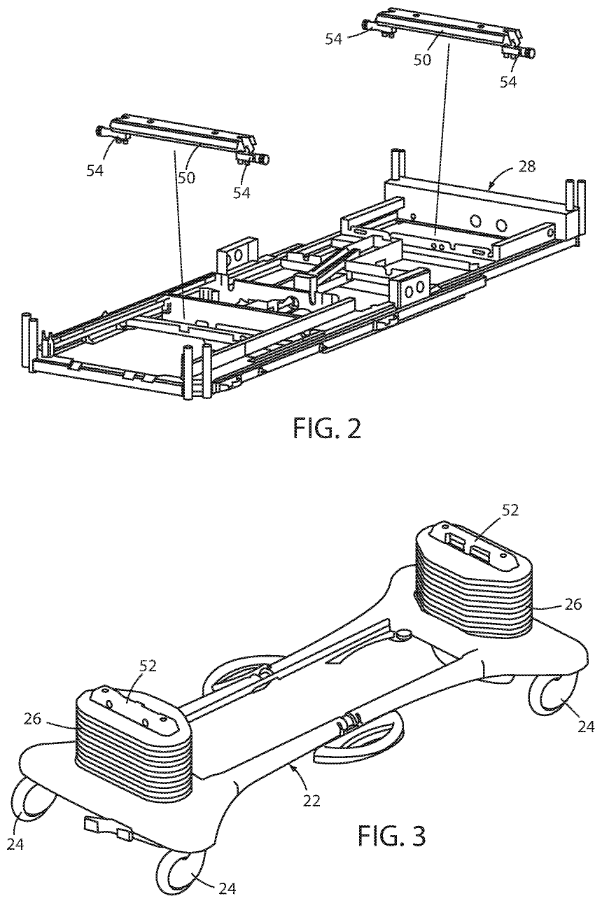

FIG. 2 illustrates in greater detail litter frame 28 separated from lifts 26 and base 22. Litter frame 28 is also shown in FIG. 2 with support deck 30 removed. Litter frame 28 is supported by two lift header assemblies 50. A first one of the lift header assemblies 50 is coupled to a top 52 (FIG. 3) of a first one of the lifts 26, and a second one of the lift header assemblies 50 is coupled to the top 52 of the second one of the lifts 26. Each lift header assembly 50 includes a pair of force sensors 54, which will be described herein as being load cells, but it will be understood that force sensors 54 may be other types of force sensors besides load cells. The illustrated embodiment of patient support apparatus 20 includes a total of four load cells 54, although it will be understood by those skilled in the art that different numbers of load cells may be used in accordance with the principles of the present disclosure. Load cells 54 are configured to support litter frame 28. More specifically, load cells 54 are configured such that they provide complete and exclusive mechanical support for litter frame 28 and all of the components that are supported on litter frame 28 (e.g. support deck 30, footboard 32, siderails 34, etc.). Because of this construction, load cells 54 are adapted to detect the weight of not only those components of patient support apparatus 20 that are supported by litter frame 28 (including litter frame 28 itself), but also any objects or persons who are wholly or partially being supported by support deck 30.

The basic mechanical construction of patient support apparatus 20, as shown in FIGS. 1-3, is the same as, or nearly the same as, the mechanical construction of the Model 3002 S3 bed manufactured and sold by Stryker Corporation of Kalamazoo, Mich. This mechanical construction is described in greater detail in the Stryker Maintenance Manual for the MedSurg Bed, Model 3002 S3, published in 2010 by Stryker Corporation of Kalamazoo, Mich., the complete disclosure of which is incorporated herein by reference. It will be understood by those skilled in the art that the basic mechanical structure of patient support apparatus 20 can be designed with other types of constructions, such as, but not limited to, those described in commonly assigned, U.S. Pat. No. 7,690,059 issued to Lemire et al., and entitled HOSPITAL BED; commonly assigned U.S. Pat. Publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION; and/or commonly assigned, U.S. Pat. No. 10,130,536 to Roussy et al., entitled PATIENT SUPPORT USABLE WITH BARIATRIC PATIENTS, the complete disclosures of all of which are also hereby incorporated herein by reference. The basic mechanical construction of patient support apparatus 20 may also take on forms different from what is disclosed in the aforementioned references.

Load cells 54 are part of an exit detection system that, when armed, issues an alert when the patient exits from patient support apparatus 20. The exit detection system is adapted to be armed via control panel 48a. After being armed, the exit detection system determines when an occupant of patient support apparatus 20 has left, or is likely to leave, patient support apparatus 20, and issues an alert and/or notification to appropriate personnel so that proper steps can be taken in response to the occupant's departure (or imminent departure) in a timely fashion. In at least one embodiment, the exit detection system monitors the center of gravity of the patient using the system and method disclosed in commonly assigned U.S. Pat. No. 5,276,432 issued to Travis and entitled PATIENT EXIT DETECTION MECHANISM FOR HOSPITAL BED, the complete disclosure of which is incorporated herein by reference. In other embodiments, the exit detection system determines if the occupant is about to exit, or already has exited, from patient support apparatus 20 by determining a distribution of the weights detected by each load cell 54 and comparing the detected weight distribution to one or more thresholds. In such embodiments, the center of gravity may or may not be explicitly calculated.

Other manners for implementing the exit detection system are also possible. These include, but are not limited to, any of the manners disclosed in the following commonly assigned patent applications: U.S. patent application Ser. No. 14/873,734 filed Oct. 2, 2015, by inventors Marko Kostic et al. and entitled PERSON SUPPORT APPARATUS WITH MOTION MONITORING; U.S. patent publication 2016/0022218 filed Mar. 13, 2014, by inventors Michael Hayes et al. and entitled PATIENT SUPPORT APPARATUS WITH PATIENT INFORMATION SENSORS; and U.S. patent application Ser. No. 15/266,575 filed Sep. 15, 2016, by inventors Anuj Sidhu et al. and entitled PERSON SUPPORT APPARATUSES WITH EXIT DETECTION SYSTEMS, the complete disclosures of all of which are incorporated herein by reference. Further, in some embodiments, load cells 54 may be part of both an exit detection system and a scale system that measures the weight of a patient supported on support deck 30. The outputs from the load cells 54 are processed, in some embodiments, in any of the manners disclosed in commonly assigned U.S. patent application Ser. No. 62/428,834 filed Dec. 1, 2016, by inventors Marko Kostic et al. and entitled PERSON SUPPORT APPARATUSES WITH LOAD CELLS, the complete disclosure of which is incorporated herein by reference.

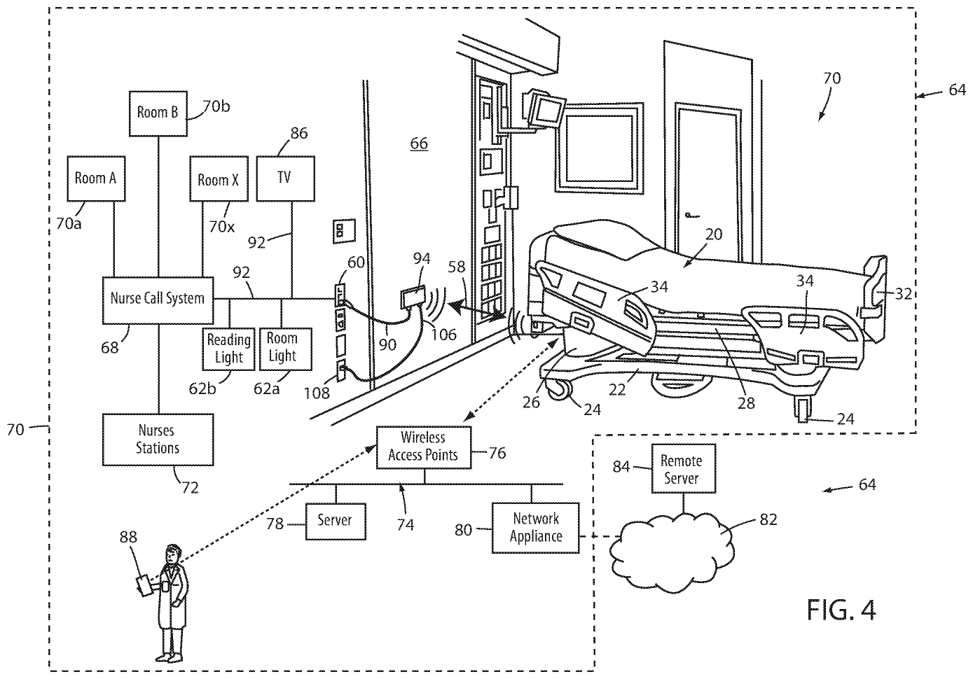

Regardless of how implemented, patient support apparatus 20 is adapted to communicate an alert when the exit detection system is armed and detects that a patient is about to, or has, exited. One manner in which the alert is communicated to a conventional nurse call system 68 is shown in FIG. 4. Patient support apparatus 20 communicates with a nurse call system 68, and vice versa, through a communication link 58 that is established between patient support apparatus 20 and a conventional headwall wall outlet 60, which is conventionally communicatively coupled to nurse call system 68. Communication link 58 is a wireless communication link in the example shown in FIG. 4, but may be a wired link in other embodiments. In addition to forwarding an exit detection alert over communication link 58 from patient support apparatus 20 to nurse call system 68, communication link 58 may be used for communicating a variety of other information.

One example of such information are the audio signals of the patient and a remotely positioned nurse. That is, a patient onboard patient support apparatus 20 is able to communicate with a remotely positioned nurse by speaking into a microphone onboard patient support apparatus 20, and patient support apparatus 20 forwards these audio signals to a remotely positioned nurse by transmitting them over communication link 58 to wall outlet 60, which, as noted, is in communication with nurse call system 68. Similarly, a remotely positioned nurse is able to speak into a microphone coupled to the nurse call system and have his/her voice signals forwarded to wall outlet 60, which are then transmitted over communication link 58 to a speaker onboard patient support apparatus 20.

FIG. 4 illustrates additional details of a typical healthcare facility 64. As shown therein, healthcare facility 64 includes a headwall 66, nurse call system 68, a plurality of rooms 70 (70a, 70b . . . 70x), one or more nurses'stations 72, a local area network 74, one or more wireless access points 76, a patient support apparatus server 78, and one or more network appliances 80 that couple LAN 74 to the internet 82, thereby enabling server 78 and other applications on LAN 74 to communicate with computers outside of healthcare facility 64, such as, but not limited to, a geographically remote server 78. Wall outlet 60 is typically electrically coupled by one or more conductors 92 to a television 86 and one or more room devices (e.g. a room light 62a, a reading light 62b, etc.). It will be understood by those skilled in the art, however, that the healthcare facility infrastructure shown in FIG. 4 may vary widely from healthcare facility to healthcare facility.

For example, patient support apparatus 20 may be used in healthcare facilities having no wireless access points 76, no connection to the internet 82 (e.g. no network appliances 80), and/or no patient support apparatus server 78. Still further, local area network 74 may include other and/or additional servers installed thereon, and/or room 70, in some healthcare facilities 64, may be semi-private room having multiple patient support apparatuses 20 and multiple wall outlets 60. Still other variations are possible. It will therefore be understood that the particular healthcare facility infrastructure shown in FIG. 4 is merely illustrative, and that patient support apparatus 20 is constructed to be communicatively coupled to healthcare facility communication infrastructures which are arranged differently from that of FIG. 4, some of which are discussed in greater detail below.

As is shown in FIG. 4, patient support apparatus 20 is adapted to be communicatively coupled to the wall outlet 60 on headwall 66 by way of a wireless communication link 58 that wirelessly couples patient support apparatus 20 to a wireless headwall unit 94. Headwall unit 94, in turn, is coupled by way of a cable 90 to wall outlet 60. Headwall unit 94 and patient support apparatus 20 are able to communicate wirelessly with each other in a bidirectional fashion. That is, messages can be wirelessly sent from patient support apparatus 20 to headwall unit 94, and messages can be wirelessly sent from headwall unit 94 to patient support apparatus 20.

Although not shown in FIG. 4, patient support apparatus 20 may further be configured to be able to communicate with wall outlet 60 via a cable, if desired. When such wired communication is desired, a nurse call cable 90 is connected directly from patient support apparatus 20 to wall outlet 60. Unless wall outlet 60 has room for two cable plugs, the end of cable 90 shown in FIG. 4 that is plugged into wall outlet 60 is removed in order to allow the cable from patient support apparatus 20 to be inserted therein. Alternatively, the end of the cable 90 shown in FIG. 4 that is plugged into headwall unit 94 may be removed and plugged into patient support apparatus 20. However attained, once a cable 90 is coupled between patient support apparatus 20 and wall outlet 60, a wired communication link 58 is established that bypasses headwall unit 94 (if present).

Wall outlet 60 is coupled to one or conductors 92 that electrically couple the wall outlet 60 to nurse call system 68 and to one or more other devices, such as television 86, room light 62a, and/or reading light 62b. Conductors 92 are typically located behind headwall 66 and not visible. In some healthcare facilities, conductors 92 may first couple to a room interface board that includes one or more electrical connections electrically coupling the room interface board to television 86 and/or nurse call system 68. Still other communicative arrangements for coupling wall outlet 60 to nurse call system 68 and television 86 are possible.

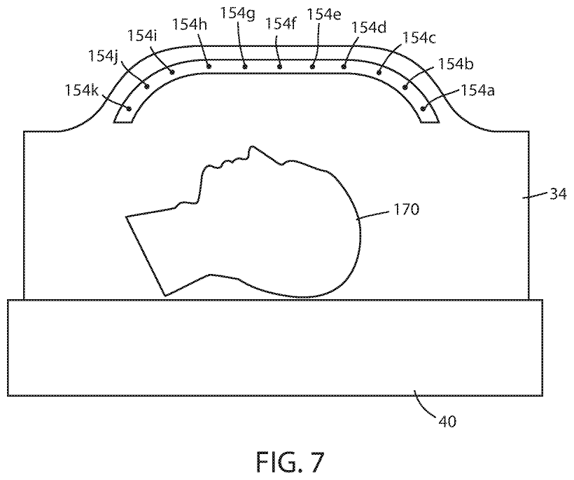

Communication link 58 (FIG. 4) enables patient support apparatus 20 to communicate with nurse call system 68, television 86, room light 62a, and/or reading light 62b. A patient supported on patient support apparatus 20 who activates a nurse call control on patient support apparatus 20 causes a signal to be conveyed via communication link 58 to the nurse call system 68, which then sends a notification to one or more remotely located nurses (e.g. nurses at one of the nurses'stations 72). If the patient uses a TV control positioned on one of the control panels (e.g. control 126k of control panel 48c; see FIG. 7) to change a channel or change the volume of television 86, the control conveys a signal along link 58 to the wall outlet 60, and the signal is thereafter passed from outlet 60 to television 86. Similarly, if the patient uses a room light or reading light control on one of the control panels, he or she is able to turn on or off the room light 62a and reading light 62b.

Outlet 60 (FIG. 4) often includes a plurality of pins (e.g. 37 pins), and the audio signals that are passed between the patient and a remotely positioned nurse are transmitted over a separate set of pins than the pins that transmits control signals for controlling television 86. Additional pins are used for communicating other information between patient support apparatus 20 and nurse call system 68 and/or other devices positioned within room 70 (e.g. television 86, room light 62a, reading light 62b)

Headwall unit 94 of FIG. 4 is adapted to wirelessly receive signals from patient support apparatus 20 and deliver the signals to wall outlet 60 in a manner that matches the way the signals would otherwise be delivered to wall outlet 60 if a conventional nurse call cable (e.g. cable 90) were connected directly between patient support apparatus 20 and wall outlet 60. In other words, patient support apparatus 20 and headwall unit 94 cooperate to provide signals to wall outlet 60 in a manner that is transparent to wall outlet 60 and nurse call system 68 such that these components cannot detect whether they are in communication with patient support apparatus 20 via wired or wireless communication. In this manner, a healthcare facility can utilize the wireless communication abilities of one or more patient support apparatuses 20 without having to make any changes to their existing wall outlet 60 or to their nurse call system 68.

In addition to sending signals received from patient support apparatus 20 to wall outlet 60, headwall unit 94 is also adapted to forward signals received from wall outlet 60 to patient support apparatus 20. Such bidirectional communication includes, but is not limited to, communicating audio signals between a person supported on patient support apparatus 20 and a nurse positioned remotely from patient support apparatus 20 (e.g. nurses'station 72). The audio signals received by headwall unit 94 from patient support apparatus 20 are forwarded to wall outlet 60, and the audio signals received by wall outlet 60 from nurse call system 68 are forwarded to one or more speakers onboard patient support apparatus 20.

Headwall unit 94 also communicates the data and signals it receives from patient support apparatus 20 to the appropriate pins of wall outlet 60. Likewise, it communicates the data and signals it receives and/or detects on the pins of wall outlet 60 to patient support apparatus 20 via wireless messages. The wireless messages include sufficient information for patient support apparatus 20 to discern what pins the messages originated from, or sufficient information for patient support apparatus 20 to decipher the information included in the message.

Headwall unit 94 (FIG. 4) includes a power cable 106 having an end adapted to be inserted into a conventional electrical outlet 108. Power cable 106 enables headwall unit 94 to receive A/C power from the mains electrical supply via outlet 108. It will be appreciated that, in some embodiments, headwall unit 94 is battery operated and cable 106 may be omitted. In still other embodiments, headwall unit 94 may be both battery operated and include cable 106 so that in the event of a power failure, battery power supplies power to headwall unit 94, and/or in the event of a battery failure, electrical power is received through outlet 108.

In at least one embodiment, headwall unit 94 includes any and/or all of the same functionality as, and/or components of, the headwall units 76 disclosed in commonly assigned U.S. patent application Ser. No. 16/215,911 filed Dec. 11, 2018, by inventors Alexander Bodurka et al. and entitled HOSPITAL HEADWALL COMMUNICATION SYSTEM, the complete disclosure of which is incorporated herein by reference. Alternatively, or additionally, headwall unit 94 may include any and/or all of the same functionality as, and/or components of, the headwall interface 38 disclosed in commonly assigned U.S. patent publication 2016/0038361 published Feb. 11, 2016, entitled PATIENT SUPPORT APPARATUSES WITH WIRELESS HEADWALL COMMUNICATION, and filed by inventors Krishna Bhimavarapu et al., the complete disclosure of which is also incorporated herein by reference. Still further, headwall unit 94 and/or patient support apparatus 20 may include any of the functionality and/or components of the headwall units 140, 140a and/or patient support apparatuses 20, 20a, and/or 20b disclosed in commonly assigned U.S. patent application Ser. No. 62/833,943 filed Apr. 15, 2019, by inventors Alexander Bodurka et al. and entitled PATIENT SUPPORT APPARATUSES WITH NURSE CALL AUDIO MANAGEMENT, the complete disclosure of which is incorporated herein by reference. Additionally, headwall unit 94 and/or patient support apparatus 20 may include any of the functionality and/or components of the headwall units 94 and/or 94a of commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which is incorporated herein by reference. In still other embodiments, patient support apparatus 20 may be configured to only be able to communicate with wall outlet 60 via a cable (i.e. not include the ability to implement a wireless communication link 58).

As was noted, patient support apparatus 20 is configured to communicate with one or more servers on local area network 74 of the healthcare facility (FIG. 4), such as patient support apparatus server 78. Patient support apparatus server 78 is adapted, in at least one embodiment, to receive status information from patient support apparatuses 20 positioned within the healthcare facility and distribute this status information to caregivers, other servers, and/or other software applications. In some embodiments, patient support apparatus server 78 is configured to communicate at least some of the status data received from patient support apparatuses 20 to remote server 84. Such communication may take place via network appliance 80 (FIG. 4), which may be a conventional router and/or a gateway that is coupled to the Internet 82. The remote server 84, in turn, is also coupled to the Internet 82, and patient support apparatus server 78 is provided with the URL and/or other information necessary to communicate with remote server 84 via the Internet connection between network 74 and server 84.

In some embodiments, any of the data communicated to patient support apparatus server 78 may be shared with one or more electronic devices 88 (FIG. 4) that are carried by caregivers and/or other appropriate personnel. Electronic devices 88 may refer to smart phones, tablet computers, and/or other devices that are able to access the local area network 74 via wireless access points 76. Electronic devices 88 may include an internal controller, memory, network transceiver, display, and one or more controls. The memory may include a software application that is executed by the internal controller and that carries out one or more functions described herein, such as, but not limited to, a remote display of data from a spectrometer of patient support apparatus 20 and/or a remote control of aspects of the spectrometer. Electronic devices 88 may also, or alternatively, include smart televisions or displays that are adapted to communicate with a computer network and that are adapted to display data received from patient support apparatus server 78, but that are not adapted to send commands and/or data back to the server 78 or patient support apparatus 20.

It will be understood that the architecture and content of local area network 74 will vary from healthcare facility to healthcare facility, and that the example shown in FIG. 4 is merely one example of the type of network a healthcare facility may be employ. Typically, additional servers, such as an ADT server, an EMR server, a caregiver assignment server, and/or still other servers will be hosted on network 74 and one or more of them may be adapted to communicate with patient support apparatus server 78 and/or electronic devices 88.

FIG. 5 depicts a block diagram of patient support apparatus 20, headwall unit 94, and a healthcare facility's local area network 74. Headwall unit 94 includes a Bluetooth transceiver 96, an IR transceiver 98, a headwall unit controller 100, configuration circuitry 102, a television controller 104, a headwall interface 110, and a unit ID 112. Bluetooth transceiver 96 is adapted to communicate with a Bluetooth transceiver 122 onboard patient support apparatus 20 using RF waves in accordance with the conventional Bluetooth standard (e.g. IEEE 802.14.1 and/or the standard maintained by the Bluetooth Special Interest Group (SIG) of Kirkland, Wash., USA). In some embodiments, transceivers 96 and 116 utilize Bluetooth Low Energy communications.

Infrared transceiver 98 is adapted to communicate with an infrared transceiver 118 positioned onboard patient support apparatus 20 using infrared waves. In some embodiments, IR transceiver 98 is configured to communicate its unit ID 112 to such patient support apparatuses via IR transceiver 98, which is a short range transceiver that is configured to only communicate with an adjacent patient support apparatus 20 when the patient support apparatus 20 is nearby (e.g. without about five to ten feet or so). Such an adjacent patient support apparatus 20 then communicates the received wall unit ID 112 along with its own unique ID 120 (FIG. 5) to server 78 which is adapted to correlate the wall unit ID 112 to a particular location with the healthcare facility. In this manner, server 78 is able to use headwall units 94 to determine the location of patient support apparatuses 20.

In some embodiments, the location of each headwall unit 94 is stored in a memory within that particular wall unit 94 and shared with the devices it communicates with (e.g. patient support apparatuses 20). In some embodiments, the location of each wall unit 94 is stored within memory 130 (FIG. 5) of each patient support apparatus 20. Still further, in some embodiments, the location of each wall unit 94 is stored within a memory accessible to server 78. Alternatively, or additionally, the location of each wall unit 94 may be stored in two or more of the aforementioned locations.

Headwall unit controller 100 (FIG. 5) is adapted to control the operation of transceivers 96, 98, configuration circuitry 102, TV controller 104, and headwall interface 110. Headwall interface 110 is adapted to change the electrical state of one or more pins that are in electrical communication with communication outlet 60 (via cable 90). Headwall interface 110 changes these electrical states in response to instructions from controller 100. For example, if an exit detection system onboard patient support apparatus 20 detects a patient exit, patient support apparatus 20 sends an exit alert signal to headwall unit 94 and controller 100 responds by instructing headwall interface 110 to change the electrical state of at least one pin that is used to signal an exit alert (or a generic priority alert) to the nurse call system 68 via communications outlet 60. In some embodiments, headwall interface 110 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCIING, the complete disclosure of which is incorporated herein by reference. Alternatively, or additionally, headwall interface 110 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Headwall unit 94 may also be configured to perform any of the functions of the headwall units 94 disclosed in the above-mentioned '778 patent application.

Configuration circuitry 102 and TV controller 104 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, headwall unit 94 may be configured to perform any of the functions of the headwall units 144 disclosed in the aforementioned '508 patent application.

Patient support apparatus 20 includes a control system 122 comprising a plurality of nodes 124a-g coupled together by an embedded network 126. Nodes 124 include a main control node 124a, a Bluetooth node 124b, a control panel node 124c, a remote communications node 124d, a motion control node 124e, an infrared node 124f, and a spectral node 124g. It will be understood that the number of nodes 124 shown in FIG. 5, as well as the function of these nodes 124, may vary, including consolidating the functionality of one or more of these nodes into fewer nodes, dividing the functionality of one or more nodes 124 into a greater number of nodes 124, adding new nodes with new functionality, and/or eliminating one or more of the nodes 124 shown in FIG. 5. It will also be understood that, in at least one embodiment, embedded network 126 is a Controller Area Network (CAN), although it will be understood that in other embodiments, a different type of embedded network may be utilized, such as, but not limited to, an onboard Ethernet, an I-Squared-C bus, a Local Interconnect Network (LIN) bus, a Firewire network, an RS-232 network, an RS-485 network, a Universal Serial Bus (USB), a Serial Peripheral Interface (SPI) bus, combinations of these, and/or in other manners.

Main node 124a includes a controller 128 and is coupled to a memory 130. Memory 130 includes the data and programming for carrying out the functions described herein. Memory 130 also includes patient support apparatus ID 120, which uniquely identifies the patient support apparatus 20 and distinguishes it from other patient support apparatuses 20. Memory 130 may be conventional flash memory, one or more hard drives, and/or any other type of non-volatile memory that is accessible by main controller 128.

Main controller 128 is configured to send ID 120 to server 78, in some embodiments, along with location information so that server 78 is able to tell which particular patient support apparatus 20 is positioned where within a particular healthcare facility. Server 78 may also use the location of the particular patient support apparatus 20 to determine which patient and/or caregiver is assigned to a particular room 70 of the healthcare facility. This determination of the assigned patient or caregiver may be carried out by communication with one or more other servers on network 74 that store data identifying which patients and/or caregivers are assigned to which locations within the healthcare facility.

Patient support apparatus 20 further includes a microphone 132 in communication with main control node 124a and controller 128. Microphone 132 is used by a patient when he or she wishes to speak to a remotely positioned nurse, as will be described in more detail below. The patient's voice is converted to audio signals by microphone 132 and controller 128 is adapted to forward these audio signals to an adjacent communications outlet 60 positioned in wall 66 (FIG. 4). When a cable 90 is coupled between patient support apparatus 20 and outlet 60, controller 128 forwards these audio signals to outlet 60 via the cable. When no such cable 90 extends between patient support apparatus 20 and outlet 60, controller 128 wirelessly forwards these audio signals to headwall unit 94 (using transceiver 116, or in some embodiments, transceiver 118) and controller 100 of headwall unit 94 forwards these audio signals to outlet 60. As was noted, outlet 60 is in electrical communication with a conventional nurse call system 68 that is adapted to route the audio signals to the correct nurse's station 76, and/or other location. In some embodiments, microphone 132 acts as both a microphone and a speaker. In other embodiments, a separate speaker may be included in order to communicate the voice signals received from the remotely positioned nurse. In some embodiments, the audio communication between patient support apparatus 20 and communications outlet 60 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

Bluetooth node 124b includes Bluetooth transceiver 116 that is adapted to communicate with the Bluetooth transceiver 96 of wall unit 94. Infrared node 124f includes infrared transceiver 118 that is adapted to communicate with infrared transceiver 98 of wall unit 94 when patient support apparatus 20 is positioned within range of wall unit 94 and oriented such that infrared transceiver 118 faces toward infrared transceiver 98. Control panel node 124c includes one or more of the control panels 48 of patient support apparatus 20. As shown in FIG. 5, control panel node 124c includes a display 134, one or more controls 136, and a display controller 138. Display controller 138 is adapted to control what is displayed on display 134 and to oversee communications between control panel node 124c and the rest of control system 122. Display 134 may be a conventional LCD screen (either touch sensitive or not), and controls 136 may comprise one or more keys, switches, buttons, and/or touch sensitive sensors that are used to command, control, and/or enter information into patient support apparatus 20.

Remote communication node 124d (FIG. 5) includes one or more network transceivers 140 and a communication controller 142. Network transceiver(s) 140 may be WiFi transceivers, Ethernet transceivers, and/or other any other type of transceiver that is capable of allowing patient support apparatus 20 to communicate with network 74 and/or a remote network that is coupled to network 74 (e.g. the Internet 82). Communication controller 142 is adapted to oversee the communications between transceiver 140 and the network 74, as well as to oversee communications between remote communication node 124d and the rest of control system 122.

Motion control node 124e includes a motion controller 144 that is in communication with one or more powered actuators, such as the lifts 26 and a Fowler actuator 146. Motion controller 144 is responsible for converting motion commands detected on embedded network 126 into motion control signals that are sent to the appropriate actuators 26, 146. Motion controller 144 may also be adapted to report the current position of one or more of the actuators 26, 146 to the other nodes 124 via the embedded network 126. Still further, motion controller 144 may oversee the communications between motion control node 124e and the rest of control system 122. Lifts 26 (FIG. 4) drive the lifts 26 up and down. Lifts 26 may be hydraulic actuators, electric actuators, or any other suitable powered device for raising and lowering litter frame 28 with respect to base 22. Lifts 26 are activated by motion controller 144 whenever a user activates one or more controls 136 that control the height of litter frame 28. Fowler actuator 146 controls the pivoting of head section 40 with respect to a horizontal axis and may also be a hydraulic, electric, or some other type of powered actuator.

Spectral node 124g includes a spectral controller 150 that is in communication with one or more emitters 152, one or more detectors 154, and a camera 156. As will be discussed in greater detail below, emitters 152 are adapted to emit electromagnetic waves that pass through a volume of space positioned adjacent the nose and mouth of the patient on the patient support apparatus 20. After passing through this volume of space, the electromagnetic waves are detected by one or more detectors 154 and the detected electromagnetic waves are spectrally analyzed by spectral controller 150. The spectral analysis may be performed to determine a variety of different parameters of the patient, such as, but not limited to, the concentration of carbon dioxide exhaled by the patient, the patient's breathing rate, the patient's metabolic rate, and/or other parameters. Spectral control node 124g, emitter(s) 152, and detector(s) 154 therefore act as a built-in spectrometer 160 of patient support apparatus 20 that is configured to perform spectral analyses of the gasses exhaled by a patient positioned on patient support apparatus 20, such as, but not limited to, carbon dioxide.

Camera 156 is included in some embodiments of patient support apparatus 20 in order to detect the current location and orientation of the patient's face so that spectral controller 150 can determine which beams of electromagnetic waves emitted by emitters 152 pass through the volume of space in front of the patient's mouth and nose. Camera 156 is positioned such that its field of view encompasses the space above head section 40 in which a patient's head is typically positioned when the patient is sitting or lying on the mattress on patient support apparatus 20. In other words, camera 156 is positioned such that its field of view encompasses the patient's head and one or more detectors 154 whenever the patient is supported on patient support apparatus 20. Although the patient support apparatus 20 shown in FIG. 5 only includes a single camera 156, it will be understood that more than one camera 156 may be included on patient support apparatus 20.

Each camera 156, in some embodiments, is a camera from the RealSense™ product family D400 series marketed by Intel Corporation of Santa Clara, Calif. For example, in some embodiments, each camera is an Intel RealSense™ D455 Depth Camera that includes two imagers, an RGB sensor, a depth sensor, an inertial measurement unit, a camera module and a vision processor. Further details regarding this camera are found in the June 2020, (revision 009; document number 337029-009) datasheet entitled “Intel® RealSense™ Product Family D400 Series,” published by Intel Corporation of Santa Clara, Calif., the complete disclosure of which is incorporated herein by reference. Other types of depth cameras marketed by the Intel Corporation, as well as other types of depth cameras marketed by other entities may also, or alternatively, be used according to the teachings of the present disclosure. In some embodiments, cameras may be used that are of the same type(s) as those disclosed in commonly assigned U.S. Pat. No. 10,368,39 issued to Derenne et al. on Jul. 20, 2019, and entitled VIDEO MONITORING SYSTEM, the complete disclosure of which is incorporated herein by reference. As will be discussed in greater detail below, the images captured by camera 156 are utilized by spectral controller 150 in order to determine which set of detector(s) 154 will yield a spectral analysis that captures the gas exhaled by the patient.

The location of camera 156 on patient support apparatus 20 may vary from embodiment to embodiment. In at least one embodiment, camera 156 is mounted in one of the head end siderails 34. Camera 156 may be either mounted in a fixed orientation, or it may be coupled to a mounting structure that allows the orientation of the camera to be automatically adjusted by spectral controller 150 such that the camera may have its field of view automatically adjusted. Still further, camera 156 may include zoom features that allow spectral controller 150 to control the zooming in and zooming out of camera 156 such that both close-up images and wider field of view images may be captured, as desired.

In some embodiments, spectral controller 150 includes commercially available software that is adapted to carry out the analysis of the images captured by camera 156. This image analysis is designed to locate the three dimensional position of the patient's nose and mouth with respect to detectors 154 and/or emitter 152 so that spectral controller 150 can spectrally analyze the beams of electromagnetic waves that pass through the volume of space in which the patient exhales. In some of these embodiments, spectral controller 150 includes the commercially available software suite referred to as OpenCV (Open Source Computer Vision Library), which is an open source computer vision library supported by Willow Garage of Menlo Park, Calif. The algorithms of the OpenCV library include a comprehensive set of computer vision and machine learning algorithm that are designed to be used to detect and recognize faces, identify objects, classify human actions in videos, track camera movements, track moving objects, extract 3D models of objects, produce 3D point clouds from stereo cameras, stitch images together to produce high resolution images of entire scenes, find similar images from an image database, follow eye movements, recognize scenery and establish markers to overlay scenery with augmented reality, and other tasks.

In order to assist spectral controller 150 in determining the current location of the patient's head on patient support apparatus 20, a memory (not shown) accessible to controller 150 may be provided in which is stored various attribute data regarding patient support apparatus 20. This attribute data may define, for example, the color, size, shape, location, and/or other data of those portions of the patient support apparatus 20 that are positioned within the field of view of camera 156. Such attribute data may therefore assist spectral controller 150 in identifying within the images captured by camera 156 the siderail(s) 34, the mattress, the head section 40, features of these components, and/or other components of patient support apparatus 20. The identification of these components may assist controller 150 in identifying the pixels of the captured images that correspond to the patient's face, as well as the pixels that do not correspond to the patient' face.

Controllers 128, 138, 142, 144, and 150, as well as any other controller described herein, may take on a variety of different forms. In the illustrated embodiment, each of these controllers is implemented as a conventional microcontroller. However, these controllers may be modified to use a variety of other types of circuits—either alone or in combination with one or more microcontrollers—such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. In some embodiments, the functions of one or more of these controllers may be consolidated into a single controller or a smaller number of controllers. The instructions followed by the controllers disclosed herein when carrying out the functions described herein, as well as the data necessary for carrying out these functions, are stored in a corresponding memory that is accessible to that particular controller (e.g. memory 130 for controller 128)

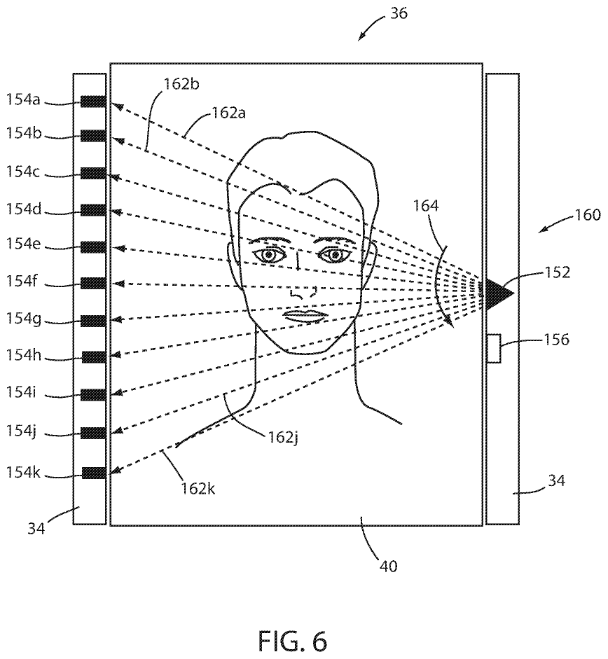

The operation of spectrometer 160 may be more easily understood with reference to FIG. 6, which shows the emitter 152 and detectors 154 of spectrometer 160 coupled to the right and left head end siderails 34 of patient support apparatus 20. It will be understood that, although FIG. 6 shows emitter 152 coupled to the left head siderail 34 (from the patient's perspective when lying on his or her back) and detectors 154 coupled to the right head siderail 34, the position of emitter 152 and detectors 154 may be swapped. Additionally, emitter(s) 152 and/or detectors(s) 154 may be positioned at other locations on patient support apparatus 20, such as, but not limited to, a headboard (not shown), head section 40, and/or other locations.

Emitter 152 (FIG. 6) is adapted to emit beams 162 of electromagnetic waves across the patient support apparatus 20 from one head end siderail 34 to an oppositely positioned siderail 34. In the embodiment shown in FIG. 6, emitter 152 is adapted to emit beams 162 in a sweeping motion indicated by arrow 164. That is, emitter 152 is adapted to first emit a beam 162a toward detector 154a, then move to detector 154b, 154c, etc. Although FIG. 6 refers to each beam 162a-k with a separate letter designation, it will be understood that, in at least some embodiments, emitter 152 may emit a continuous beam 162 that simply sweeps across the detectors 154a-k. Alternatively, emitter 152 may be configured to first emit a beam 162a to detector 154a, stop the emission, then emit a second beam 162b to detector 154b, and so on. Still further, in some embodiments, multiple emitters 152 may be present, such as, but not limited to, one emitter 152 for each detector 154, in which the emitters 152 do not have to change the aiming direction of the electromagnetic waves that they emit.

In some embodiments, emitter 152 is configured to emit infrared waves. In other embodiments, emitter 152 may be configured to emit a laser. It will be understood that emitter 152 may be configured to emit still other types of electromagnetic waves, including, but limited to, visible light waves, ultraviolet waves, and/or waves that belong to other portions of the electromagnetic spectrum. In general, emitter 152 is configured to emit electromagnetic waves that can be spectrally analyzed (after detection by one or more detectors 154) by spectral controller 150 to determine the absolute and/or relative concentration of carbon dioxide in the gas exhaled by the patient. As will be discussed in greater detail below, by monitoring the amount of carbon dioxide in the gas exhaled by the patient, spectral controller 150 and/or another controller onboard patient support apparatus 20 (and/or servers 78 and/or 84) can determine one or more parameters about the patient, such as, the patient's breathing rate, metabolic rate, whether the patient is asleep or awake, and/or other parameters.

Detectors 154 may be any commercially available sensors that are able to detect a spectrum of the wavelengths emitted by the emitter(s) 152 and determine the absorption, scattering, and/or other quantities resulting from the emitted waves passing through the gas exhaled by the patient. In other words, detectors 154 may be any commercially available sensors used in conventional spectrometers.

In some embodiments, spectral controller 150 is configured to take one or more calibration readings when a patient is not present on the patient support apparatus 20. These calibration readings are taken to establish baseline readings of the amount of carbon dioxide that is present in the air between the emitter(s) 152 and the detector(s) 154. These baseline readings are then used by spectral controller 150 when the patient is present to determine differences in the carbon dioxide levels that are due to the patient's breathing. In some embodiments, spectral controller 150 may be configured to subtract the baseline reading from the readings obtained when the patient is present, thereby leaving only those changes in the readings that are due to the patient's respiration. The baselines reading may also, or alternatively, be used in other manners to calibrate and/or process the readings captured when the patient is present on the patient support apparatus.

In some embodiments, spectral controller 150 is configured to automatically take one or more of these calibration readings when the patient is not on patient support apparatus 20. In some of these embodiments, main controller 128 is configured to monitor the outputs from force sensors 54 to determine whether a patient is present on patient support apparatus 20 or not. When main controller 128 detects that no patient is present (and/or detects when a patient exits), it sends a message to spectral controller 150 informing it that the patient is not present (or has just exited). In such embodiments, spectral controller 150 may be configured to automatically take one or more calibration readings from detector(s) 154 in response to such messages from main controller. In this manner, the caregiver does not need to take any manual steps to calibrate spectrometer 160. Instead, spectrometer 160 self-calibrates itself when no patient is present on patient support apparatus 20.

In some embodiments, spectral controller 150 may be configured to automatically take the aforementioned calibration readings periodically when no patient is present on patient support apparatus 20. For example, in some embodiments, spectral controller 150 is configured to take a calibration reading as soon as, or shortly after, the patient exits patient support apparatus, and then periodically thereafter (e.g. every 1 hour or so) take additional calibration readings for as long as the patient is off of the patient support apparatus 20. Other time periods (besides 1 hour or so) may, of course, alternatively be used. The purpose of the calibration readings is to detect variations in the ambient levels of carbon dioxide (and/or any other gasses of interest that are to be monitored by spectrometer 160) so that, when the patient is present, spectral controller 150 knows what the ambient levels of carbon dioxide (and/or other gasses) are, and can therefore determine how those ambient levels are being affected by the patient's breathing.

In some embodiments, instead of, or in addition to, utilizing signals from force sensors 54 to determine if a patient is present or not on patient support apparatus 20, spectral controller 150 may be adapted to process the outputs from camera 156 to determine if a patient is present or not. In such embodiments, spectral controller 150 may be configured to automatically perform one or more calibration readings when no patient is detected.

In addition to, and/or in lieu of, receiving a signal from main controller 128 indicating that no patient is present on patient support apparatus 20 (as detected by load cells 54), spectral controller 150 may be configured to receive a weight reading from main controller 128 indicating the weight of the patient. That is, in some embodiments, main controller 128 is configured to utilize force sensors 54 to implement a scale function in which the weight of the patient is automatically determined when the patient is positioned on patient support apparatus 20. In some embodiments, this automatic determination of the patient's weight may be carried out in any of the manners disclosed in commonly assigned U.S. patent application Ser. No. 63/255,223 filed Oct. 13, 2021, by inventors Sujay Sukumaran et al. and entitled PATIENT SUPPORT APPARATUS WITH PATIENT WEIGHT MONITORING, the complete disclosure of which is hereby incorporated herein by reference.

The weight of the patient conveyed by main controller 128 to spectral controller 150 is used, in some embodiments, to compute a metabolic rate of the patient. The computed metabolic rate may be estimated based on the patient's weight and rate of carbon dioxide exhalation. That is, in some embodiments, spectral controller 150 may be configured to estimate the level of carbon dioxide being exhaled by the patient that is due to respiration (i.e. the amount over the baseline amount in the ambient air), and compute an approximate metabolic rate based on the patient's weight. The metabolic rate may be determined in joules per hour per kilogram of body weight, or using some other units. In an alternative embodiment, spectral controller 150 forwards its measurement of the carbon dioxide exhaled by the patient (over the ambient levels), or a similar measurement, to main controller 128 and main controller 128 computes the estimate of the patient's metabolic rate. That is, in some embodiments, the computation of the patient's metabolic rate may be performed by other controllers besides spectral controller 150. In still other embodiments, remote communication node 124d may forward the patient weight readings and carbon dioxide level readings to an off-board source, such as server 78, which then remotely computes an estimate of the patient's metabolic rate.

In addition to, or in lieu of, determining the metabolic rate of the patient, spectral controller 150 may be configured to determine the respiration rate of the patient based on the spectral analysis it performs on the signals detected by detector(s) 154. In such embodiments, spectral controller 150 is configured to continuously, or nearly continuously (e.g. multiple times a second) take readings from the detector(s) 154 and spectrally analyze the results. The result will indicate that the carbon dioxide levels detected by spectrometer 160 rise and fall in synchronization with the patient's breathing. Therefore, by monitoring the rising and falling of the carbon dioxide levels, spectral controller 150 is able to determine the respiration rate of the patient.

In any of the embodiments discussed herein, any one or more of the controllers onboard patient support apparatus 20 may be configured to issue an alert if any of the parameters monitored by spectrometer 160 exceed an upper threshold and/or decrease below a lower threshold. For example, in some embodiments, main controller 128 is configured to issue an alert if the patient's breathing rate exceeds a high rate, if the patient's breathing rate falls below a lower rate, if the patient's metabolic rate exceeds a high threshold, if the patient's metabolic rate falls below a lower rate, if the patient's exhaled carbon dioxide levels exceed a high level, and/or if the patient's exhaled carbon dioxide levels fall below a lower level. In such embodiments, the alert may be a local alert that is confined to patient support apparatus 20 itself (e.g. the illumination one or more lights on patient support apparatus 20, the display of one or more messages or icons on display 124, and/or the emission of one or more sounds from patient support apparatus 20), and/or the alert may be sent to a remote device, such as a message sent by node 124d to patient support apparatus server 78. In some embodiments, patient support apparatus server 78 is further configured to forward the alert to one or more of the electronic devices 88 that are associated with the caregiver(s) who is assigned to the particular patient support apparatus 20 from which the alert originated.

In some embodiments, patient support apparatus 20 may be configured by a caregiver, or other user, as to the limits for any of the upper or lower rates or thresholds for the alerts mentioned above. That is, in some embodiments, an authorized person may utilize control panel 48a to enter limits for when an alert should be issued for the patient's respiration rate, metabolic rate, and/or carbon dioxide levels. Patient support apparatus 20 may further be configured to allow a user to remotely configure these limits, such as through one or more of the electronic devices 88 that are in communication with patient support apparatus server 78. In such embodiments, after the user enters the desired limit(s) into his or her electronic device 88, the device sends the limits to patient support apparatus server 78, which in turn forwards the limit(s) to patient support apparatus 20. Main controller 128 responds by monitoring the corresponding parameter and issuing an alert when the parameter goes past the limit (e.g. exceeds an upper limit or falls below a lower limit).

In some embodiments, main controller 128 and/or one of the other controllers onboard patient support apparatus 20 may be configured to utilize the outputs of spectrometer 160 and camera 156 (and/or force sensors 54) to automatically determine if the patient is asleep or not. Such determinations may be made based on the amount of movement of the patient, as well as changes in the respiration rate of the patient. The amount of movement of the patient may be determined through automatic processing of images from camera 156 and/or through processing of the readings from the force sensors 54 (which, as noted, may be configured to monitor the movement of the patient, such as, but not limited to, calculating the center of gravity of the patient). The amount of carbon dioxide exhaled by the patient may also be monitored for determining when the patient is asleep. When the patient's respiration rate changes to lower level (and/or when the patient's exhaled carbon dioxide changes to a lower level), and the drop in that level coincides with a reduced, or limited, amount of patient movement, one or more of the controllers onboard patient support apparatus 20 may conclude that the patient is asleep. In such embodiments, controller 128 may be configured to send a notification to an off-board device, such a patient support apparatus server 78 and/or one or more of the electronic devices 88 indicating that the patient has fallen asleep. Conversely, one or more controllers onboard patient support apparatus 20 may use a reverse process to automatically determine when the patient wakes up, and to, if configured to do so, send a notification to patient support apparatus server 78 and/or one or more electronic devices 88 indicating that the patient has awakened (or is awake).

In some embodiments, spectral controller 150 is configured to determine which one, or ones, of the multiple beams 162 to spectrally analyze based on images captured from camera 156. This is better understood with respect to FIG. 7. FIG. 7 illustrates a potential relative position of a patient's head 170 to a plurality of detectors 154 integrated into one of the head end siderails 34. The vantage point of FIG. 7 is similar to the vantage point that camera 156 may possess in some embodiments. Thus, when camera 156 captures an image similar to what is shown in FIG. 7, spectral controller 150 is configured in some embodiments to determine the relative position of the patient's mouth and nose to the array of detectors 154a-k. Based on this determination, spectral controller 150 is configured to select a set of one or more detectors 154a-k whose outputs it will spectrally analyze to determine the one or more parameters mentioned herein.

In the example show in FIG. 7, the patient's nose and mouth are positioned closest to detectors 156g and 156f, and controller 150 may be configured to include these detectors 156g and 156f within the set of detectors whose outputs it spectrally analyzes. Further, because the patient's nose is oriented such that, as he or she exhales, the exhaled gases will travel both upwardly from head section 40 and toward foot end 38 of patient support apparatus 20, spectral controller 150 may be configured to also include within the set of detectors 154 whose outputs it spectrally analyzes detectors 154h, 154i, 154j and 154k. Spectral controller 150 will not, in some embodiments, utilize the outputs from detectors 154e, 154d, 154c, 154b, or 154a when the patient's head is positioned and oriented relative to these detectors in the manner shown in FIG. 7.

In some embodiments, when spectral controller 150 determines that it will not analyze the outputs from one or more detectors 154, spectral controller 150 is configured to change the aim of the electromagnetic waves emitted by emitter 152 such that those waves are not aimed toward the detectors 154 whose outputs it is not processing. Thus, in the example discussed above with respect to FIG. 7, spectral controller 150 may be configured to change the aim of the electromagnetic waves emitted by emitter 152 such that they are not aimed toward any of detectors 154a-e. In some embodiments, this may involve changing the sweeping motion of the emitter 152 such that it skips these detectors 154a-e. Alternatively, spectral controller 150 may be configured to continue to aim the emitted electromagnetic waves at all of the detectors 154 and simply ignore the outputs of those detectors that it has excluded from the aforementioned set of detectors (e.g. detectors 154a-e).

It will be understood that spectral controller 150 is configured, in some embodiments, to sum together the results of all of the detectors 154 whose outputs it spectrally analyzes. Thus, for example, if spectral controller 150 is currently analyzing the outputs of only detectors 154h and 154i, and the spectral analysis of the outputs from detector 154h are a first level, and the spectral analysis of the outputs from detector 154i are at a second level, spectral controller 150 is configured to sum together the first and second levels to determine the carbon dioxide levels. In such embodiments, spectral controller 150 may be configured such that it does not adjust the aim of the electromagnetic waves emitted by emitter 152, but instead sums together all of the outputs from the detectors 154. In such embodiments, those outputs of the detectors 154 that are not aligned with the gas exhaled by the patient will yield a carbon dioxide reading that is the same as the ambient levels, and therefore will not affect the sum of the outputs of all of the detectors 154. Indeed, in such embodiments, patient support apparatus 20 may be modified to omit camera 156 altogether.

In those embodiments of patient support apparatus 20 that use camera 156 to determine the relative position of the patient, it will be understood that camera 156 is configured to repetitively capture images of the patient and spectral controller 150 is configured to repetitively analyze those images. This allows spectral controller 150 to make essentially real time adjustment to the aiming of the electromagnetic waves from emitter 152, and/or to make essentially real time adjustments to the set of detectors 154 whose outputs it is spectrally analyzing.

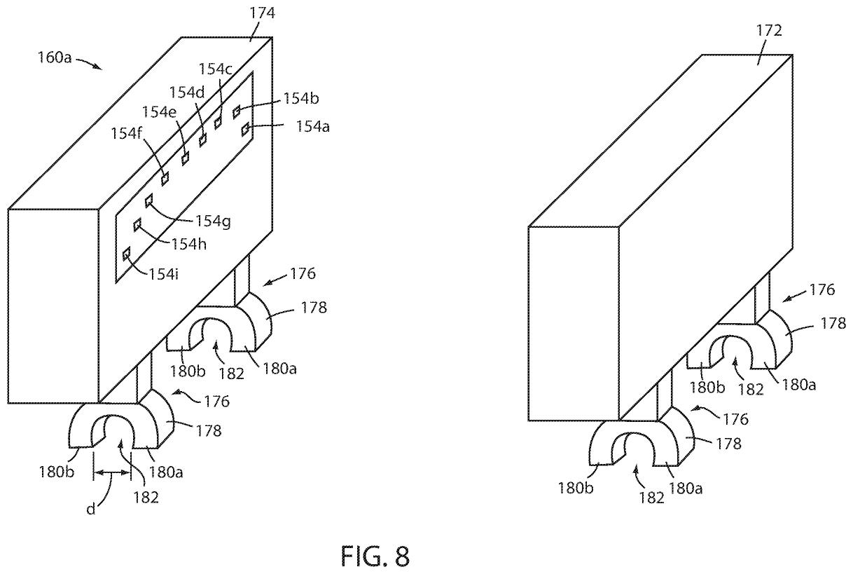

FIG. 8 illustrates an alternative spectrometer 160a that may be utilized with patient support apparatus 20 in any of the manners discussed herein. Spectrometer 160a differs from spectrometer 160 in that spectrometer 160a is adapted to be detachably coupled to a patient support apparatus 20, rather than being built into the patient support apparatus 20, like spectrometer 160. Spectrometer 160a includes an emitter structure 172 and a detector structure 174. Emitter structure 172 includes one or more emitters 152 and detector structure 174 includes one or more detectors 154. In some embodiments, emitter structure 172 may also include camera 156. Both structures 172 and 174 include a pair of attachment arms 176. Attachment arms 176 are adapted to allow structures 172 and 174 to be releasable attached to the head end siderails of a patient support apparatus that does not include a spectrometer 160 already built therein.

Attachment arms 176 may take on a wide variety of different forms so long as they allow a user to easily couple and uncouple the structures 172 and 174 to opposite siderails 34 of a patient support apparatus. In the example shown in FIG. 8, each attachment arm 176 includes a circular portion 178 that terminates at an inside end 180a and an outside end 180b. The ends 180a and 180b are separated by a distance d. Circular portion 178 is made of a flexible material (e.g. a flexible plastic) that allows the distance d to increase when attachment arm 176 is pushed onto a siderail 34 of a patient support apparatus. In other words, each siderail 34 includes a tubular upper member that has a circular diameter approximately equal to, or slightly larger than (in order to ensure a tight fit), the diameter of a circular space 182. Circular space 182 is defined by the interior of circular portion 178. Thus, as the tubular upper member of the siderail 34 is initially inserted into circular space 182, the ends 180a and 180b flex apart (distance d increases) in order to allow the tubular upper member to be received in circular space 182. Once the tubular upper member of the siderail 34 is completely positioned inside space 182, the ends 180a and 180b flex back toward each other and secure the corresponding structure 172 or 174 to the top of a siderail 34 of the patient support apparatus. When the user wishes to remove structure 172 or 174 from the siderail 34, he or she simply lifts up on the structure 172 or 174, and the upward force creates a camming force component that urges ends 180a and 180b apart sufficiently to allow the tubular upper member of the siderail 34 to exit the interior space 182, thereby decoupling the structure 172 or 174 from the corresponding siderail 34.

Spectrometer 160a (FIG. 8) includes a spectral controller 150 that operates in any of the same manners described previously with respect to spectrometer 160. Spectral controller 150 of spectrometer 160a may be positioned inside of emitter structure 172 or inside of detector structure 174 (or have portions positioned inside of both). Spectral controller 150 communicates with emitter(s) 152 and detector(s) 154 in any suitable manner. In some embodiments, each structure 172 and 174 includes a wired or wireless transceiver that allows direct communication between structures 172 and 174, as well as the internal components of these structures. One example of such an embodiment is a spectrometer 160a in which each structure 172 and 174 includes a Bluetooth transceiver and these transceivers are able to pair with each other and wirelessly communicate with each other. Other types of wireless or wired transceivers may, of course, be used.

In some embodiments of spectrometer 160a, one or both of structures 172 and 174 may include a wired or wireless transceiver that directly communicates with the associated patient support apparatus. For example, in some embodiments, one or both of structures 172 and/or 174 may include a Bluetooth transceiver that is adapted to communicate with a Bluetooth transceiver onboard the patient support apparatus (e.g. Bluetooth transceiver 116). In some embodiments, each structure 172 and 174 may include one or more cable ports for receiving one or more cables. In some of these embodiments, a single cable may be coupled at its first end to a cable port on structure 172 and at its second end to a cable port on structure 174, thereby allowing structure 172 to communicate by wired communications directly with structure 174. Alternatively, or additionally, a cable may be coupled at its first end to the port on structure 172 or 174 and at its second end to a cable port on the patient support apparatus. Still further, another cable may be coupled to at one end to a second port on the patient support apparatus and its opposite end to the cable port on the other structure 172 or 174 so that both structures 172 and 174 have a cable connecting themselves to the patient support apparatus. Still other variations are possible.

Regardless of the manner in which structures 172 and 174 communicate with each other and the patient support apparatus to which they are attached, such communication allows spectrometer 160a to carry out any of the functions described above. For example, this communication allows spectrometer 160a to receive weight information from the main controller 128 of the patient support apparatus (as detected by force sensors 54). Spectrometer 160a may use this weight information in any of the manners discussed above (e.g. to determine patient presence/absence, to calculate metabolic rate, to determine movement levels for deciding whether the patient is asleep or awake, etc.). Additionally, by being able to communicate directly with the patient support apparatus 20, spectral controller 150 is able to send alert information directly to the patient support apparatus, which is then able to forward the alert information to patient support apparatus server 78 (via communication node 124d), which in turn may be configured to forward the alert information to one or more electronic devices 88).

The patient support apparatus to which spectrometer 160a is adapted to be coupled may be exactly the same as patient support apparatus 20 with two exceptions: (1) it does not include a spectrometer 160 built therein, and (2) it includes one or more wired or wireless transceivers for communicating with spectrometer 160a (which, in some embodiments, may be a transceiver that is already incorporated into the patient support apparatus, such as transceiver 116). Because such patient support apparatuses do not include a built-in spectrometer 160, the shape of the head end siderails 34 may also be varied from the shape shown in FIG. 1. Thus, for example, the patient support apparatuses that don't include spectrometer 160 may include head end siderails that have a shape that is the same as, or similar to, the shape of the head end siderails of the patient support apparatuses 20 disclosed in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which has already been incorporated herein by reference. Spectrometer 160a may be configured to attach to, and communicate with, still other types of patient support apparatuses.

In some embodiments, patient support apparatus 20 and/or spectrometer 160a may be configured to automatically associate the outputs of the spectrometer 160 or 160a with a particular patient so that the caregiver doesn't have to manually take any steps to make such an association. Alternatively, or additionally, patient support apparatus 20 and/or spectrometer 160a may be configured to allow a server, such as server 78 and/or 84, to automatically associate the outputs of the spectrometer 160 or 160a with a particular patient so that the caregiver doesn't have to manually take any steps to make this association. Whether this automatic association is carried out by a patient support apparatus, spectrometer 160a, or a server, this automatic association allows the data from the spectrometer 160 or 160a to be automatically forwarded to the correct patient record in an electronic medical record system. Alternatively, or additionally, this also allows any alerts or other information from the spectrometer 160, 160a to be forwarded to only those electronic devices 88 that are associated with the particular caregiver(s) who is/are assigned to that particular patient. The manner in which this automatic association of spectrometer data to a particular patient, as well as this automatic selection of which electronic devices 88 to forward spectrometer information to will now be discussed in greater detail.

As was mentioned above, patient support apparatus 20 is adapted to receive a unique location ID 112 from headwall unit 94 when the patient support apparatus 20 is positioned adjacent to the headwall unit 94. A mapping of the location of each of the headwall units 94 within a particular healthcare facility is stored in a memory accessible to patient support apparatus server 78. This mapping correlates each unique location ID 112 to a particular location within the healthcare facility. After patient support apparatus 20 receives this unique location ID 112 from an adjacent headwall unit 94, it sends this unique location ID, along with its own patient support apparatus ID 120, to patient support apparatus server 78. Patient support apparatus server 78 uses the mapping to determine the location in the healthcare facility of the corresponding location ID 112, and then concludes that that particular patient support apparatus 20 is located in that same location of the healthcare facility because the patient support apparatus 20 is only able to receive the location ID 112 when it is positioned in close proximity to the headwall unit 94 that stores that location ID 112.

Patient support apparatus server 78 is therefore able to determine the location of each of the patient support apparatuses 20 within the corresponding healthcare facility. In addition, because each spectrometer 160 or 160a communicates its data to the patient support apparatus 20 to which it is coupled (either built-into, as in spectrometer 160, or detachably coupled to, as in spectrometer 160a), the location of each spectrometer 160 or 160a is also determinable by patient support apparatus server 78 (i.e. each spectrometer 160 or 160a is in the same location as its associated patient support apparatus). Each patient support apparatus 20, in some embodiments, is configured to forward spectrometer data (from its associated spectrometer 160 or 160a) to patient support apparatus server 78 via network transceiver 140. Because the patient support apparatus 20 includes within this spectrometer data the unique ID 120 of the patient support apparatus 20, server 78 concludes that the spectrometer 160, 160a is in the same location as the associated patient support apparatus 20. In some embodiments, each spectrometer 160, 160a may also have its own unique ID, and patient support apparatus 20 may include this unique ID in the spectrometer data that it forwards to server 78.

Patient support apparatus server 78 (FIG. 4), in some embodiments, is configured to communicate with one or more other servers on network 74 that contain data that associates a particular location within the healthcare facility, such as a room number and/or a bay identifier within a particular room, to either a particular caregiver and/or to a particular patient. To the extent patient support apparatus server 78 communicates with a server that associates a particular room or bay location to a particular caregiver, it is also configured to communicate with one or more servers on the network 74 that associates each caregiver to a particular patient. Thus, server 78 is configured to communicate with one or more conventional servers on network 74 that identify which particular patient is assigned to a particular room and/or bay within a room. Using this information, server 78 is able to automatically identify which patient is associated with each patient support apparatus 20, as well as which patient is associated with each spectrometer 160 or 160a. Patient support apparatus server 78 can then use this patient association data to automatically forward data from the spectrometer and/or patient support apparatus 20 to the corresponding patient's records of an Electronic Medical Records server that is in communication with network 74.

Additionally, or alternatively, patient support apparatus 20 can use the association of a particular room with one or more particular caregivers (which it gets from one or more conventional servers on network 74) to determine which electronic devices 88 are associated with those particular caregivers. This is performed through communication with a server on network 74 that stores an association between particular electronic devices 88 and particular caregivers. In some embodiments, a table of this association may be stored internally within patient support apparatus 20. Wherever stored, patient support apparatus server 78 uses this information to decide which electronic devices 88 to send notifications/alerts to. For example, if an alert is generated in room X by a spectrometer 160, patient support apparatus server 78 will receive this alert from the patient support apparatus 20 in room X and will forward the corresponding alert to only those electronic devices 88 that are associated with the caregivers who are assigned to care for the patient in room X. In this manner, alerts and notifications are targeted to only the caregivers who are responsible for the particular patient to whom the alert pertains.

Further details regarding manners in which patient support apparatus server 78 may automatically determine which caregivers and/or which patients are assigned to particular rooms and/or bays of rooms are disclosed in commonly assigned U.S. Pat. No. 11,062,585, which issued to Thomas Durlach et al. on Jul. 13, 2021, and which is entitled PATIENT CARE SYSTEM, the complete disclosure of which is incorporated herein by reference. In some embodiments, patient support apparatus server 78 may include any or all of the functionality of the patient support apparatus server 132 disclosed in the '585 patent, and/or may communicate with any of the servers disclosed in the '585 patent.

As was noted previously, in addition to data generated from spectrometer 160 and/or 160a, patient support apparatus 20 may be configured to send other data to patient support apparatus server 78. Such other data includes, but is not limited to, the status of any of its siderails 34, the status of its brake, the height of litter frame 28, the state of its exit detections system (armed/disarmed), and/or any other data (including data generated from camera 156).

In some embodiments, one or more of the electronic devices 88 may be communal electronic devices that are intended to be viewed by multiple caregivers, such as all caregivers that are assigned to a particular wing, department, unit, or some other segment of the healthcare facility. When the healthcare facility includes such communal electronic devices 88, server 78 is programmed with, or is programmed to have access to, data that lists the rooms that are associated with each such communal electronic device 88. Thus, for example, a first communal electronic device 88 may be intended to display data for rooms 400 through 440, while a second communal electronic device 88 may be intended to display data for rooms 450 through 490. In such a case, server 78 is informed of the room assignments for each communal electronic device 88 and thus only sends patient support apparatus data and/or spectrometer data from a particular room to the communal electronic device(s) 88 that are intended to display data for that particular room.

In any of the embodiments disclosed herein, server 78 may be configured to additionally execute a caregiver assistance software application of the type described in the following commonly assigned patent applications: U.S. patent application Ser. No. 62/826,097, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application Ser. No. PCT/US2020/039587 filed Jun. 25, 2020, by inventors Thomas Durlach et al. and entitled CAREGIVER ASSISTANCE SYSTEM, the complete disclosures of which are all incorporated herein by reference. When server 78 is configured to additionally execute a caregiver assistance software application of this type, the caregiver assistance software application may be modified to include data generated from spectrometer 160 and/or 160a.

In those embodiments where spectral controller 150 and/or main controller 128 (or some other controller) are adapted to determine the patient's respiration rate using the spectral analysis of the detector(s) 154, patient support apparatus 20 may be configured to implement an auxiliary sensing system for determining the patient's respiration rate. In such embodiments, patient support apparatus 20 is adapted to use two independent techniques for determining the patient's breathing rate: the spectrometer 160 or 160a, and an auxiliary method.

In some of these embodiments, main controller 128 may be adapted to forward the breathing rate determinations from both spectrometer 160, 160a and the auxiliary technique to patient support apparatus server 78 (which in turn may forward this information to one or more electronic devices 88). In other embodiments, main controller 128 may be configured to compare the breathing rates determined from each of the two methods (a first method using the spectrometer 160 or 160a and a second method using the auxiliary technique). If the rates match, main controller 128 forwards the breathing rate to patient support apparatus server 78. If they do not match, main controller 128 may be configured to not send either of the breathing rates, but instead send an error message or a notification message indicating that the patient's breathing rate is currently unable to be determined. Alternatively, in the case of a mismatch between the breathing rates, main controller 128 may be configured to send one or both of the non-matching breathing rates to server 78.

In some embodiments, patient support apparatus 20 uses the outputs of force sensors 54 as the auxiliary manner of determining the patient's breathing rate. In such embodiments, the movement of the patient's lungs and chest during breathing are sensed by force sensors 54 and processed by main controller 128 to determine the patient's breathing rate. Further details of one manner of sensing the patient's breathing rate using force sensors 54 are disclosed in commonly assigned U.S. Pat. No. 7,699,784, which issued on Apr. 20, 2010, to inventors David Wan Fong et al. and entitled SYSTEM FOR DETECTING AND MONITORING VITAL SIGNS, the complete disclosure of which is incorporated herein by reference.

In other embodiments, patient support apparatus 20 uses the outputs from camera 156 and/or one or more other cameras as the auxiliary method for determining the patient's breathing rate. In such embodiments, the boundaries of the patient's torso are detected in the capture images and the movement of these boundaries is monitored to determine the patient's breathing rate. Further details regarding one manner in which the patient's breathing rate may be determined from the analysis of captured images is disclosed in commonly assigned U.S. patent application Ser. No. 63/218,053 filed Jul. 2, 2021, by inventors Krishna Bhimavarapu et al. and entitled PATIENT VIDEO MONITORING SYSTEM, the complete disclosure of which is incorporated herein by reference.

In still other embodiments, patient support apparatus 20 may use the outputs from pressure and/or depth sensors positioned inside of the mattress positioned on patient support apparatus 20 as the auxiliary method for determining the patient's breathing rate. In such embodiments, the movement of the patient's torso causes changes in the air pressure inside of a set of bladders positioned within the air mattress, and/or causes changes in the depth to which the patient sinks into the mattress. These changes are sensed by the corresponding sensors and forwarded to main controller 128 for processing. Further details regarding one manner in which main controller 128 may determine the patient's breathing rate from pressure and/or depth sensors positioned inside of a mattress are disclosed in commonly assigned U.S. Pat. No. 10,682,273, which issued Patrick Lafleche et al. on Jun. 16, 2020, and which is entitled INFLATABLE MATTRESS AND CONTROL METHODS, the complete disclosure of which is incorporated herein by reference.

In some embodiments of spectrometer 160a, spectrometer 160a may be modified to include one or more ultra-wideband transceivers that communicate with one or more ultra-wideband transceivers built into the patient support apparatus to which the spectrometer 160a is to be coupled. In such embodiments, the patient support apparatus may be configured to automatically determine the position of the spectrometer relative to the patient support apparatus using the ultra-wideband transceivers and, if the spectrometer 160a is located within a predetermined volume of space, automatically associate the spectrometer 160a to that patient support apparatus. In other words, both spectrometer 160a and the patient support apparatus to which it is to be coupled may be constructed in accordance with the medical devices and patient support apparatuses, respectively disclosed in any of the following commonly assigned patent applications: U.S. patent application Ser. No. 63/161,175 filed Mar. 15, 2021, by inventors Krishna Bhimavarapu et al. and entitled EXERCISE DEVICE AND PATIENT SUPPORT APPARATUS; U.S. patent application Ser. No. 63/193,777 filed May 27, 2021, by inventors Thomas Deeds et al. and entitled SYSTEM FOR ASSOCIATING MEDICAL DEVICE DATA; and U.S. patent application Ser. No. 63/245,289 filed Sep. 17, 2021, by inventors Madhu Thota et al. and entitled PATIENT SUPPORT APPARATUS COMMUNICATION AND LOCATION SYSTEM, the complete disclosure of all of which are incorporated herein by reference. In such embodiments, either or both structures 172 and 174 of spectrometer 160a may include an ultra-wideband tag integrated therein.

Although camera 156 has primarily described herein as being adapted to capture visible light images, it is to be understood that, in at least some embodiments, one or more of cameras 156 may be modified to include infrared image sensing devices, either in lieu of, or in addition to, their visual light image sensors. When equipped with one or more of such infrared image sensing devices, camera 156 may be able to capture images of the patient's head and its surrounding environment (included detector(s) 154) even when the room is dark. The capturing of such infrared images utilizes existing ambient infrared light within the room, in some embodiments, and in other embodiments, utilizes one or more sources of infrared light that are provided as part of patient support apparatus 20. In addition to capturing images in dark or low-light conditions, utilizing one or more infrared cameras 156 also allows the capturing of thermal images. Such thermal images, and/or data derived from such thermal images, may be forwarded to server 78 and/or electronic devices 88.

It will also be understood that, although detectors 154 have been shown in the accompanying drawings as being formed in a contiguous array, this arrangement may be modified substantially. Thus, for example, instead of an array of detectors 154 that are positioned side by side in a generally head end 36 to foot end 38 direction (such as is shown in the attached drawings), detectors 154 may be positioned such that one or more of them are positioned above each other, either partially or wholly, in a vertical direction. Still other arrangements are possible.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus comprising:
a frame;
a support surface supported on the frame and adapted to support a patient thereon;
an emitter coupled to a first location on the patient support apparatus and adapted to emit electromagnetic waves;
a detector coupled to a second location on the patient support apparatus and adapted to detect the electromagnetic waves emitted by the emitter; and
a controller adapted to perform a spectral analysis of the electromagnetic waves detected by the detector to determine a parameter associated with gas exhaled by the patient when the patient is positioned on the patient support apparatus.

2. The patient support apparatus of claim 1 wherein the parameter is at least one of the following: a level of carbon dioxide exhaled by the patient; a breathing rate of the patient; or a metabolic rate of the patient.

3. The patient support apparatus of claim 1 further comprising a camera in communication with the controller, wherein the controller is further adapted to process an image captured by the camera to determine a relative position of the patient's face to the detector.

4. The patient support apparatus of claim 1 wherein the controller is further adapted to take calibration readings from the detector when no patient is present on the patient support apparatus.

5. The patient support apparatus of claim 4 further comprising a plurality of force sensors adapted to detect a weight of the patient when the patient is positioned on the support surface, and wherein the controller is further adapted to not take the calibration readings if patient weight is detected by the plurality of force sensors.

6. The patient support apparatus of claim 2 further comprising a plurality of force sensors adapted to detect a weight of the patient when the patient is positioned on the support surface, and wherein the parameter is the metabolic rate of the patient and the controller is further adapted to use the weight of the patient when determining the metabolic rate.

7. The patient support apparatus of claim 6 further comprising a transceiver adapted to communicate with a remote server, and wherein the controller is adapted to transmit an alert to the remote server if the metabolic rate is less than a threshold.

8. The patient support apparatus of claim 1 further comprising a location receiver adapted to receive a location ID from a fixed locator unit positioned within a healthcare facility in which the patient support apparatus is located, the location receiver adapted to receive the location ID when the patient support apparatus is positioned adjacent the fixed locator unit, and wherein the location ID is sent from the fixed locator unit to the location receiver via infrared waves, and the controller is adapted to transmit the location ID and the parameter to a remote server.

9. The patient support apparatus of claim 1 wherein the first location is a first siderail positioned adjacent a first side of the support surface, and the second location is a second siderail positioned adjacent a second side of the support surface.

10. The patient support apparatus of claim 4 wherein the controller is adapted to determine a baseline level of carbon dioxide in a sample of air near the patient support apparatus from the calibration readings, and the controller is further adapted to use the baseline level of carbon dioxide in the air sample to determine a level of carbon dioxide exhaled by the patient when the patient is positioned on the patient support apparatus.

11. A spectrometer adapted to be attached to, and communicate with, a patient support apparatus, the spectrometer comprising:
an emitter adapted to be coupled to a first location on the patient support apparatus and adapted to emit electromagnetic waves;
a detector adapted to be coupled to a second location on the patient support apparatus and adapted to detect the electromagnetic waves emitted by the emitter;
a transmitter; and
a controller adapted to perform a spectral analysis of the electromagnetic waves detected by the detector to determine a parameter associated with gas exhaled by a patient when the patient is positioned on the patient support apparatus, the controller further adapted to use the transmitter to send data to a receiver integrated into the patient support apparatus.

12. The spectrometer of claim 11 wherein the parameter is at least one of the following: a level of carbon dioxide exhaled by the patient; a breathing rate of the patient; or a metabolic rate of the patient.

13. The spectrometer of claim 11 further comprising a camera in communication with the controller, wherein the controller is further adapted to process an image captured by the camera to determine a relative position of the patient's face to the detector.

14. The spectrometer of claim 11 comprising a receiver adapted to receive a signal from the patient support apparatus indicating that no patient weight is detected on the patient support apparatus, wherein the controller is further adapted to take calibration readings from the detector when no patient is detected on the patient support apparatus and to not take the calibration readings if patient weight is detected by the patient support apparatus.

15. The spectrometer of claim 12 comprising a receiver adapted to receive a signal from the patient support apparatus indicating a weight of the patient on the patient support apparatus, and wherein the parameter is the metabolic rate of the patient and the controller is further adapted to use the weight of the patient when determining the metabolic rate.

16. The spectrometer of claim 11 further comprising a plurality of detectors, and wherein the controller is further adapted to select at least one of the plurality of detectors based on a relative position of the patient's face to the detector, and wherein the controller is further adapted to perform the spectral analysis on the electromagnetic waves detected by the selected at least one of the detectors.

17. The spectrometer of claim 11 wherein the first location is a first siderail positioned adjacent a first side of the patient support apparatus, and the second location is a second siderail positioned adjacent a second side of the patient support apparatus.

18. The spectrometer of claim 17 further comprising a plurality of detectors adapted to be coupled to the second siderail, and wherein the emitter is adapted to change an aim of the electromagnetic waves such that the electromagnetic waves are aimed at different ones of the plurality of detectors at different times, and wherein the electromagnetic waves are infrared waves.

19. The spectrometer of claim 14 wherein the controller is adapted to determine a baseline level of carbon dioxide in a sample of air near the patient support apparatus from the calibration readings, and to use the baseline level of carbon dioxide in the air sample to determine a level of carbon dioxide exhaled by the patient when the patient is positioned on the patient support apparatus.

20. The spectrometer of claim 11 wherein the data sent to the patient support apparatus includes at least one of the parameter or a unique identifier of the spectrometer.
